(19) United States
(12) Patent Application Publication
(10) Pub. No.: US 2006/0194319 A1
(43) Pub. Date: Aug. 31, 2006

(54) Promoter substitution for immunoglobulin therapy

(76) Inventor: Carol F. Webb, Oklahoma City, OK (US)

Correspondence Address: FULBRIGHT & JAWORSKI L.L.P., 600 CONGRESS AVE., SUITE 2400, AUSTIN, TX, 78701, US

(21) Appl. No.: 11/219,347

(22) Filed: Sep. 2, 2005

Related U.S. Application Data
(60) Provisional application No. 60/606,701, filed on Sep. 2, 2004.

(51) Int. Cl. C12N 15/87; C12N 15/86; C12N 5/08

(52) U.S. Cl. 435/372; 435/456; 435/455

(57) ABSTRACT

The present invention involves the identification of Bright as involved in immunoglobulin production, and the targeting of that function for the treatment of disease states associated with pathologic immunoglobulin production. Also provided are methods of identifying candidate substances with Bright-inhibitory activity.

This application claims benefit of priority to U.S. Provisional Application Ser. No. 60/606,701, filed Sep. 2, 2004, the entire contents of which are hereby incorporated by reference.

The government owns rights in the application pursuant to funding form the National Institutes of Health, Grant Nos. AI044215 and A145864.

### BACKGROUND OF THE INVENTION

1. Field of the Invention

The present invention relates generally to the fields of immunology and molecular biology. More particularly, it concerns ability of certain immunoglobulin (Ig) promoters, in particular murine Ig promoters, to function differentially. Thus, reagents derived from these promoters will find use in treating various disease states that arise from lack of Ig.

2. Description of Related Art

Antibodies, also known as immunoglobulins (Ig), form a critical part of the human immune response. These large, bivalent receptor-like molecules, produced by B lymphocytes, are found both on cell surfaces and free in body fluids. Thanks to a complicated genetic system of gene rearrangement and somatic hypermutation, the human antibody repertoire is vast, with B cells capable of producing antibodies that bind to an almost endless array of self and non-self antigens. In some cases, the binding of the antigen alone may be sufficient, impacting the ability of the antigen to perform its detrimental function. In other contexts, the antibodies mark the antigen for further removal or destruction by other immune cells (phagocytes, T-cells, etc.), or by the complement cascade.

Given their central role in the immune response, it is not surprising that the absence of immunoglobulin product can have devastating effects. For example, X-linked agammaglobulinemia (XLA) is an inherited immunodeficiency disease caused by mutations in the enzyme Bruton's tyrosine kinase (Btk). The gene for Btk is on the X chromosome and the disease affects approximately 1 in every 300,000 males all over the world. Therefore, females who have two copies of the Btk gene are generally healthy, but are carriers for the disease who may have sons with only one defective Btk enzyme. XLA patients typically exhibit less than 0.1 percent of the normal numbers of B lymphocytes in their blood, and antibody production is low to absent. This is the result of a block in B cell development at the early pro-B to pre-B cell stage in the bone marrow.

### SUMMARY OF THE INVENTION

Thus, in accordance with the present invention, there is provided a human Ig transcription cassette comprising, in a 5′ to 3′ arrangement (a) a promoter comprising a TATA element; (b) at least two human Ig variable heavy (V_{H}) segments; (c) at least two human Ig diversity (D) segments; (d) at least two human Ig heavy joining (J_{H}) segments; and (e) a human Ig constant heavy (C_{H}) segment. The transcription cassette may comprise ten D segments. The transcription cassette may comprise six J_{H} segments. The CH segment may be Cμ or Cγ. The promoter may be a murine Ig promoter, such as a J558 family promoter.

The transcription cassette is comprised within a vector, such as a non-viral vector (e.g., a plasmid, a phagemid or a cosmid) or a viral vector (e.g., an adenoviral vector, a retroviral vector, an adeno-associated viral vector, a herpesviral vector, or a vaccinia viral vector). The transcription cassette may further comprise a transcription termination signal, and/or a selectable or screenable marker segment operably linked to said promoter.

Also provided is a method of converting a human lymphocytic progenitor cell into a B cell comprising transforming said B cell with a first transcription cassette comprising, in a 5′ to 3′ arrangement (a) a promoter comprising a TATA element; (b) at least two human Ig variable heavy (V_{H}) segments; (c) at least two human Ig diversity (D) segments; (d) at least two human Ig heavy joining (J_{H}) segments; and (e) a human Ig constant heavy (C_{H}) segment. Transferring may comprise homologous recombination of said transcription cassette into the genome of said lymphocytic progenitor cell.

The lymphocytic progenitor cell may be obtained from a human subject prior to transforming, and is reintroduced into said human subject after transforming, for example, from a human subject suffers from primary agammaglobulinemia, such as X-linked agammaglobulinemia, X-linked agammaglobulinemia with growth hormone deficiency, and autosomal recessive agammaglobulinemia. The lymphocytic progenitor cell may be obtained from cord blood or bone marrow. The lymphocytic progenitor cell may be obtained from cord blood or bone marrow of one subject and introduced, after transformation, into a genetically-related subject.

The method may further comprising transforming said lymphocytic progenitor cell with a second transcription cassette comprising, in a 5′ to 3′ arrangement (a) a promoter comprising a TATA element; (b) at least two human Ig variable heavy (V_{H}) segments; (c) at least two human Ig diversity (D) segments; (d) at least two human Ig heavy joining (J_{H}) segments; and (e) a human Ig constant heavy (C_{H}) segment, wherein said V_{H} segments are distinct from those in said first transcription cassette.

As used herein the specification, “a” or “an” may mean one or more. As used herein in the claim(s), when used in conjunction with the word “comprising”, the words “a” or “an” may mean one or more than one. As used herein, “another” may mean at least a second or more.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1—Extracts from CL01, a B-cell line, were immunoprecipitated with anti-Btk (C20), anti-TFII-I, anti-Br and control GtIg and immunoblotted for the presence of TFII-I, Bright and Btk proteins.

FIG. 2—Bright/Btk/TFII-I complexes bind Bright sites within a B cell line. Anti-Bright, anti-Btk, anti-TFII-I or control goat antibodies (GtIg) were used in modified chromatin immunoprecipitation experiments with lysates of the B cell line, BCg3R-1d, and the T cell hybridoma, KD3B5.8. Immunoprecipitated DNA was PCR amplified at final dilutions of 1:100, 1:500 and 1:1000 (represented by triangles) for the presence of the IgH V1 promoter. Ten percent of the DNA used for each immunoprecipitation was used as a positive control (Input).

FIG. 3-Bright was transfected into Raji cells, a B-cell line that does not express Bright. Anti-Bright antibody was used to immunoprecipitate Bright and associated proteins. Blots were developed for Bright and TFII-I.

FIGS. 4A-B—(FIG. 4A) A standard curve for IgH DNA was generated by Real Time PCR using triplicate CT values from four experiments. (FIG. 4B) Cos-7 cells were transfected with Bright, Btk, TFII-I expression vectors and an IgH reporter plasmid. Ig mRNA levels were quantitated by Real Time PCR.

FIG. 5—IgH mRNA was measured in triplicate samples from Cos-7 cells expressing Bright, Btk, TFII-I/p70 and an IgH promoter construct using a standard curve (FIG. 4A).

### DESCRIPTION OF ILLUSTRATIVE EMBODIMENTS

As discussed above, there is a great need for improved methods of treating autoimmune disorders, particularly those that are associated with reductions in immunoglobulin (Ig) production. The inventor's previous studies have demonstrated a link between Bruton's tyrosine kinase (Btk), Bright and X-linked immunodeficiency disease (Webb et al., 2000). Others have proposed that defects in Btk can be overcome by Btk gene therapy. However, data indicate that Btk has multiple roles in the B cell, leading to concerns that overexpression of this protein may have undesirable effect via signaling through other pathways.

Since mice also develop mutations in Btk, but the related immunodeficiency observed in such animals is less severe, the inventor examined the regulatory signals of the 13 Ig heavy chain families. It was observed that at least one of these families, which is used extensively in the adult animal, contains a strong TATA consensus sequence. Since most of the 50 functional human Ig heavy chain families contain TATA-less promoters that are dependent on the Btk-Bright pathway, it was hypothesized that the loss of Bright function results in a more complete block in human Ig production.

Thus, the present invention approaches Ig deficiency diseases not by modulation of trans-acting molecules such as Btk, but by altering the cis-acting regulatory signals that control Ig expression. The inventor proposes that the use of murine or murine-like promoters in cells that lack functional Btk and/or Bright molecules will circumvent the absence of this transcriptional activation pathway. In one embodiment, the invention comprises use of Ig mini-locus expression cassettes for the transfer and expression of rearranged or partially rearranged Ig genes. Both site specific and non-specific integration into recipient cells, both ex vivo and in vivo, are contemplated. Various details of the invention are discussed below.

I. Bright-Independent Promoter Structure

1. Bruton's Tyrosine Kinase and Bright

The transcription factor Bright (B cell regulator of IgH transcription) is a member of a growing family of proteins that interact with DNA through a highly conserved A+T-rich interaction domain, or ARID (Herrscher et al., 1995). Currently, Bright is the only mammalian member of this family for which target sequences have been identified, and which binds to DNA in a sequence-specific fashion. ARID family proteins include the Drosophila proteins Dead ringer and eyelid that play important roles in lineage decisions in the gut and eyelid of the fruit fly, and are required for embryonic segmentation (Gregory et al., 1996; Treisman et al., 1997); retinoblastoma binding protein (Rbp1) that interacts with retinoblastoma protein in a cell cycle-specific fashion (Fattaey et al., 1993); and BDP, a ubiquitously expressed human protein identified in a two-hybrid screen as a novel protein that also interacts with retinoblastoma protein (Rb) (Numata et al., 1999). The yeast protein SWI/1 has homology to Bright, and is a component of a larger protein complex that serves to modulate chromatin organization in that organism (Peterson and Herskowitz, 1992; Burns and Peterson, 1997). Likewise, the human SWI-SNF complex contains a 270 kDa protein with non-sequence specific DNA binding activity that is also a member of the ARID family (Dallas et al., 2000). Thus, members of this family may participate in lineage decisions, cell cycle control, tumor suppression and modulation of chromatin. These functions are not mutually exclusive and may result from overlapping mechanisms.

Most ARID family proteins are expressed ubiquitously. However, murine Bright expression is largely limited to adult cells of the B lymphocyte lineage where its expression is tightly regulated and is restricted at the mRNA level to the pre-B cell and peanut agglutinin-high germinal center cell populations (Herrscher et al., 1995; Webb et al., 1991; Webb et al., 1998). Activated splenic B cells in the mouse can be induced to express Bright after antigen binding, but the protein is not present in the majority of peripheral IgM⁺ B cells (Webb et al., 1991; Webb et al., 1998). Induction of Bright expression in B cell lines or in mature activated B lymphocytes using lipopolysaccharide or antigen results in upregulation of IgH transcription approximately 3- to 6-fold above basal levels (Herrscher et al., 1995; Webb et al., 1991; Webb et al., 1989). Transcriptional activation is tightly associated with DNA binding sites 5′ of some VH promoters or within the intronic E1 enhancer.

Bright binding sites associated with the intronic Eμ enhancer also function as matrix-association regions, or MARs, A+T rich regions that have been proposed to organize chromatin into transcriptionally active domains (Herrscher et al. 1995; Webb et al., 1991). NFμNR (nuclear factor μ negative regulator) is another MAR-binding protein complex that binds DNA sequences overlapping Bright binding sites. NFμNR contains the ubiquitously expressed CAAAT displacement protein (CDP/Cut/Cux) (Wang et al., 1999). While non-B cells in the mouse express NFμNR, B lymphocytes generally do not exhibit such protein complexes. These data have led to the hypothesis that Bright and NFμNR play opposing roles in regulating the immunoglobulin locus (Webb et al., 1999). Transfection studies in which Bright and CDP were coexpressed showed repression of Bright (Wang et al., 1999). Therefore, Bright may activate transcription, directly or indirectly through chromatin remodeling or through more complex interactions with additional proteins. NFμNR may act in opposition to that activity (Wang et al., 1999).

The inventor has determined that Bruton's tyrosine kinase, or Btk, associates with Bright in activated murine B lymphocytes (Webb et al., 2000). Btk is an X-linked gene that encodes a tyrosine kinase critical for proper development and maintenance of B lymphocytes both in humans and in mice (reviewed in (Conley et al., 1994; Satterthwaite and Witte, 1996). Defects in this enzyme account for 90% of the severe B cell immunodeficiencies in man, and result in X-linked agammaglobulinemia (XLA), an immunodeficiency state characterized by blocks at the pro-B cell stage of development and severely depressed serum antibody levels (Conley et al., 1994). Although Btk is clearly the defective gene product in both human and murine diseases, the molecular mechanisms by which Btk deficiencies result in blocks in B cell development are currently unknown. X-linked immunodeficient (xid) mice, the mouse model for XLA, produce a mutated Btk protein that fails to form stable complexes with Bright (Webb et al., 2000).

The inventor has characterized the human Bright homologue and determined its expression in B lymphocyte subpopulations. Bright was cloned from a human B cell library and the sequence was determined to be identical to that published previously as Dril 1 (Kortschak et al., 1998). Although these studies suggested that Dril 1, or human Bright, mRNA was expressed in multiple tissues (Kortschak et al., 1998), protein and DNA binding activity were not investigated. The inventor's data indicate that Bright/Dril 1 mRNA may be expressed in a smaller number of tissues than previously thought. Furthermore, these data demonstrate that the human protein effectively binds the Bright prototype sequence motif. Investigation of sorted B cell subpopulations demonstrated that human Bright expression was similar in many ways to expression of the murine homologue; although, Bright mRNA was expressed at slightly earlier stages of normal B cell development in man than in the mouse. On the other hand, expression of Bright protein in human transformed cell lines differed dramatically from that observed in the mouse. Finally, results reveal that human Bright and Btk associate to form DNA-binding complexes, with which may further involve the Btk substrate TFII-I.

2. Murine TATA-Containing Promoters

A promoter is a DNA sequence recognized by the synthetic machinery of the cell, or introduced synthetic machinery, required to initiate the specific transcription of a gene. The phrase “under transcriptional control” means that the promoter is in the correct location and orientation in relation to the nucleic acid to control RNA polymerase initiation and expression of the gene.

The term promoter generally refers to a group of transcriptional control modules that are clustered around the initiation site for RNA polymerase II. Much of the thinking about how promoters are organized derives from analyses of several viral promoters, including those for the HSV thymidine kinase (tk) and SV40 early transcription units. These studies, augmented by more recent work, have shown that promoters are composed of discrete functional modules, each consisting of, on average, 7-20 bp of DNA, and containing one or more recognition sites for transcriptional activator or repressor proteins.

At least one module in each promoter functions to position the start site for RNA synthesis. The best known example of this is the TATA box, but in some promoters lacking a TATA box, such as the promoter for the mammalian terminal deoxynucleotidyl transferase gene and the promoter for the SV40 late genes, a discrete element overlying the start site itself helps to fix the place of initiation.

Additional promoter elements regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either co-operatively or independently to activate transcription.

Of the 40 functional human Ig genes described by Inaba et al. (1998) only 5 members of the VH1 family had consensus TATA boxes and heptamer/octamer spacing similar to that found in the prototypic J558 family gene from the BCL1 tumor described in Buchanan et al. (1997). The VH1 family is underexpressed relative to other human VH families. Therefore, the inventor proposes, in one embodiment, to construct a mini-locus with VH3-23 and VH4 family members that contain the prototypic mouse J558 promoter. This construct will contain the consensus sequence TAAATAT beginning at −31 base pairs relative to the transcription start sites of the VH genes. Nineteen base pairs upstream, the consensus heptamer and nonamer sequences (CTCAGA-2 bp-ATGCAAT) with a two base pair spacer will be inserted as spacing between nonamer and heptamer elements affected transcription efficiency in vitro (Buchanan et al., 1995). At least 150 bases of 5′ flanking and promoter sequence will be used in each case. For example, the native BCL1 sequence to be used will be SEQ ID NO:1:

5′-aaagtgtcccttcttctgaagcagtagtaagtccttatgtaagatgt
accctgtctcatgaatatgcaaatcaggtgagtctatggtggTAAATATa
gggatatctacacacctcaaaaacttaagatcacagtagtctctacagtc
acaggagtacac-3′

The J558 family is the largest VH family in the mouse, with as many as 50 members. VH families are defined by their sequence homology and that sequence homology generally extends well into the promoter and 5′ flanking sequence. Thus, various other TATA-containing promoters and their flanking sequences may be utilized in accordance with the present invention.
II. Human Ig Heavy Chain Mini-Locus

Tuaillon et al. (1993) described a human Ig heavy-chain mini-locus that permits recombinatorial rearrangement similar to that seen in fetal/pre-immune repertoires. Constructs contained two heavy-chain variable regions, 10 diversity segments, 6 heavy-chain joining segments and either Cμ or Cμ+Cγ constant segments. Seventy transcripts were cloned and sequenced following rearrangement. Thus, the authors concluded that a significant antibody repertoire could be generated from cells transformed with such constructs.

Such mini-loci, under the transcriptional control of strong TATA-containing promoters in accordance with the present invention, may be introduced into cells using any suitable method of gene transfer, including both non-viral and viral means (discussed in the following section). The following is a discussion of the various elements of such constructs.

1. Human Ig Variable Heavy Segments

Variable heavy (V_{H}) segments, for human Ig genes, number on the order of 75-100 (Matsuda et al., 1998). These segments provide the basis for antibody diversity as part of the rearrangement process and each is associated with its own individual promoter. These segments are located upstream of the D segments, and are each associated with a discrete leader sequence that permits their translation.

2. Human Ig Diversity Segments

Heavy chain diversity segments (D_{H}), numbering about 30, provide a physical link between the V_{H} segment and the downstream sequences in the Ig heavy chain mRNA. However, their primary role is to generate additional diversity in the antigen binding region of the antibody.

3. Human Heavy Joining Segments

The six heavy chain joining segments (J_{H}) provide a mechanism for linking the variable/diversity portion of the Ig gene to the constant region, as well as making up part of the antigen binding coding region. The first step in heavy chain rearrangement is the joining of D_{H} and J_{H}. Subsequently, the resulting joined DJ segment is rearranged to bring it into proximity of the appropriate V_{H} region.

4. Human Ig Constant Heavy Segments

The heavy chain constant regions (CH) define the class of the antibody being produced, and include C_{μ}, C_{δ}, C_{γ3}, C_{γ1}, C_{γ2b}, C_{γ2a}, C_{ε} and C_{α}. Unlike the rearrangement process that eliminates unneeded V_{H}, D_{H} and J_{H} segments, the ultimate selection of C_{H} is made by virtue of differential RNA processing.

III. Vectors and Vector Delivery

As discussed above, expression cassettes encoding human Ig mini-locus will be utilized to express Ig in target cells. Expression vectors are genetic constructs that provide appropriate signals for the propagation and proper expression of sequences therein. Elements designed to optimize messenger RNA stability and translatability in host cells may also be included.

1. Non-Promoter Regulatory Elements

As discussed above, the present invention will rely on the use of promoters that do not require functional Bright molecules for activation. However, other regulatory elements may be provided to enhance or control gene expression.

For example, enhancers are genetic elements that increase transcription from a promoter located at a distant position on the same molecule of DNA. Enhancers are organized much like promoters. That is, they are composed of many individual elements, each of which binds to one or more transcriptional proteins.

The basic distinction between enhancers and promoters is operational. An enhancer region as a whole must be able to stimulate transcription at a distance; this need not be true of a promoter region or its component elements. On the other hand, a promoter must have one or more elements that direct initiation of RNA synthesis at a particular site and in a particular orientation, whereas enhancers lack these specificities. Promoters and enhancers are often overlapping and contiguous, often seeming to have a very similar modular organization.

2. Polyadenylation Signals

One will typically desire to include a polyadenylation signal to effect proper polyadenylation of the gene transcript. The nature of the polyadenylation signal is not believed to be crucial to the successful practice of the invention, and any such sequence may be employed such as human growth hormone and SV40 polyadenylation signals. Also contemplated as an element of the expression cassette is a terminator. These elements can serve to enhance message levels and to minimize read through from the cassette into other sequences.

3. Selectable Markers

In certain embodiments of the invention, the cells contain nucleic acid constructs of the present invention, a cell may be identified in vitro or in vivo by including a marker in the expression construct. Such markers would confer an identifiable change to the cell permitting easy identification of cells containing the expression construct. Usually the inclusion of a drug selection marker aids in cloning and in the selection of transformants, for example, genes that confer resistance to neomycin, puromycin, hygromycin, DHFR, GPT, zeocin and histidinol are useful selectable markers. Alternatively, enzymes such as herpes simplex virus thymidine kinase (tk) or chloramphenicol acetyltransferase (CAT) may be employed. Immunologic markers also can be employed. The selectable marker employed is not believed to be important, so long as it is capable of being expressed simultaneously with the nucleic acid encoding a gene product. Further examples of selectable markers are well known to one of skill in the art.

4. Viral Expression Vectors

In certain embodiments of the invention, the expression construct comprises a virus or engineered construct derived from a viral genome. The ability of certain viruses to enter cells via receptor-mediated endocytosis, to integrate into host cell genome and express viral genes stably and efficiently have made them attractive candidates for the transfer of foreign genes into mammalian cells (Ridgeway, 1988; Nicolas and Rubenstein, 1988; Baichwal and Sugden, 1986; Temin, 1986). The first viruses used as gene vectors were DNA viruses including the papovaviruses (simian virus 40, bovine papilloma virus, and polyoma) (Ridgeway, 1988; Baichwal and Sugden, 1986) and adenoviruses (Ridgeway, 1988; Baichwal and Sugden, 1986). These have a relatively low capacity for foreign DNA sequences and have a restricted host spectrum. Furthermore, their oncogenic potential and cytopathic effects in permissive cells raise safety concerns. They can accommodate only up to 8 kB of foreign genetic material but can be readily introduced in a variety of cell lines and laboratory animals (Nicolas and Rubenstein, 1988; Temin, 1986).

A. Adenovirus

One of the preferred methods for gene delivery involves the use of an adenovirus expression vector. “Adenovirus expression vector” is meant to include those constructs containing adenovirus sequences sufficient to (a) support packaging of the construct and (b) to express an antisense polynucleotide that has been cloned therein. In this context, expression does not require that the gene product be synthesized.

The expression vector comprises a genetically engineered form of adenovirus. Knowledge of the genetic organization of adenovirus, a 36 kB, linear, double-stranded DNA virus, allows substitution of large pieces of adenoviral DNA with foreign sequences up to 7 kB (Grunhaus and Horwitz, 1992). In contrast to retrovirus, the adenoviral infection of host cells does not result in chromosomal integration because adenoviral DNA can replicate in an episomal manner without potential genotoxicity. Also, adenoviruses are structurally stable, and no genome rearrangement has been detected after extensive amplification. Adenovirus can infect virtually all epithelial cells regardless of their cell cycle stage. So far, adenoviral infection appears to be linked only to mild disease such as acute respiratory disease in humans.

Adenovirus is particularly suitable for use as a gene transfer vector because of its mid-sized genome, ease of manipulation, high titer, wide target cell range and high infectivity. Both ends of the viral genome contain 100-200 base pair inverted repeats (ITRs), which are cis elements necessary for viral DNA replication and packaging. The early (E) and late (L) regions of the genome contain different transcription units that are divided by the onset of viral DNA replication. The E1 region (E1A and E1B) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression and host cell shut-off (Renan, 1990). The products of the late genes, including the majority of the viral capsid proteins, are expressed only after significant processing of a single primary transcript issued by the major late promoter (MLP). The MLP (located at 16.8 m.u.) is particularly efficient during the late phase of infection, and all the mRNA's issued from this promoter possess a 5′-tripartite leader (TPL) sequence which makes them preferred mRNA's for translation.

In a current system, recombinant adenovirus is generated from homologous recombination between shuttle vector and provirus vector. Due to the possible recombination between two proviral vectors, wild-type adenovirus may be generated from this process. Therefore, it is critical to isolate a single clone of virus from an individual plaque and examine its genomic structure.

Generation and propagation of the current adenovirus vectors, which are replication deficient, depend on a unique helper cell line, designated 293, which was transformed from human embryonic kidney cells by Ad5 DNA fragments and constitutively expresses E1 proteins (Graham et al., 1977). Since the E3 region is dispensable from the adenovirus genome (Jones and Shenk, 1978), the current adenovirus vectors, with the help of 293 cells, carry foreign DNA in either the E1, the D3 or both regions (Graham and Prevec, 1991). In nature, adenovirus can package approximately 105% of the wild-type genome (Ghosh-Choudhury et al., 1987), providing capacity for about 2 extra kb of DNA. Combined with the approximately 5.5 kb of DNA that is replaceable in the E1 and E3 regions, the maximum capacity of the current adenovirus vector is under 7.5 kb, or about 15% of the total length of the vector. More than 80% of the adenovirus viral genome remains in the vector backbone and is the source of vector-borne cytotoxicity. Also, the replication deficiency of the E1-deleted virus is incomplete.

Helper cell lines may be derived from human cells such as human embryonic kidney cells, muscle cells, hematopoietic cells or other human embryonic mesenchymal or epithelial cells. Alternatively, the helper cells may be derived from the cells of other mammalian species that are permissive for human adenovirus. Such cells include, e.g., Vero cells or other monkey embryonic mesenchymal or epithelial cells. As stated above, the preferred helper cell line is 293.

Racher et al. (1995) disclosed improved methods for culturing 293 cells and propagating adenovirus. In one format, natural cell aggregates are grown by inoculating individual cells into 1 liter siliconized spinner flasks (Techne, Cambridge, UK) containing 100-200 ml of medium. Following stirring at 40 rpm, the cell viability is estimated with trypan blue. In another format, Fibra-Cel microcarriers (Bibby Sterlin, Stone, UK) (5 g/l) is employed as follows. A cell inoculum, resuspended in 5 ml of medium, is added to the carrier (50 ml) in a 250 ml Erlenmeyer flask and left stationary, with occasional agitation, for 1 to 4 h. The medium is then replaced with 50 ml of fresh medium and shaking initiated. For virus production, cells are allowed to grow to about 80% confluence, after which time the medium is replaced (to 25% of the final volume) and adenovirus added at an MOI of 0.05. Cultures are left stationary overnight, following which the volume is increased to 100% and shaking commenced for another 72 h.

Other than the requirement that the adenovirus vector be replication defective, or at least conditionally defective, the nature of the adenovirus vector is not believed to be crucial to the successful practice of the invention. The adenovirus may be of any of the 42 different known serotypes or subgroups A-F. Adenovirus type 5 of subgroup C is the preferred starting material in order to obtain the conditional replication-defective adenovirus vector for use in the present invention. This is because Adenovirus type 5 is a human adenovirus about which a great deal of biochemical and genetic information is known, and it has historically been used for most constructions employing adenovirus as a vector.

As stated above, the typical vector according to the present invention is replication defective and will not have an adenovirus E1 region. Thus, it will be most convenient to introduce the polynucleotide encoding the gene of interest at the position from which the E1-coding sequences have been removed. However, the position of insertion of the construct within the adenovirus sequences is not critical to the invention. The polynucleotide encoding the gene of interest may also be inserted in lieu of the deleted E3 region in E3 replacement vectors, as described by Karlsson et al. (1986), or in the E4 region where a helper cell line or helper virus complements the E4 defect.

Adenovirus is easy to grow and manipulate and exhibits broad host range in vitro and in vivo. This group of viruses can be obtained in high titers, e.g., 10⁹-10¹² plaque-forming units per ml, and they are highly infective. The life cycle of adenovirus does not require integration into the host cell genome. The foreign genes delivered by adenovirus vectors are episomal and, therefore, have low genotoxicity to host cells. No side effects have been reported in studies of vaccination with wild-type adenovirus (Couch et al., 1963; Top et al., 1971), demonstrating their safety and therapeutic potential as in vivo gene transfer vectors.

Adenovirus vectors have been used in eukaryotic gene expression (Levrero et al., 1991; Gomez-Foix et al., 1992) and vaccine development (Grunhaus and Horwitz, 1992; Graham and Prevec, 1991). Recently, animal studies suggested that recombinant adenovirus could be used for gene therapy (Stratford-Perricaudet and Perricaudet, 1991; Stratford-Perricaudet et al., 1990; Rich et al., 1993). Studies in administering recombinant adenovirus to different tissues include trachea instillation (Rosenfeld et al., 1991; Rosenfeld et al., 1992), muscle injection (Ragot et al., 1993), peripheral intravenous injections (Herz and Gerard, 1993) and stereotactic inoculation into the brain (Le Gal La Salle et al., 1993).

B. Retroviruses

The retroviruses are a group of single-stranded RNA viruses characterized by an ability to convert their RNA to double-stranded DNA in infected cells by a process of reverse-transcription (Coffin, 1990). The resulting DNA then stably integrates into cellular chromosomes as a provirus and directs synthesis of viral proteins. The integration results in the retention of the viral gene sequences in the recipient cell and its descendants. The retroviral genome contains three genes—gag, pol, and env—that code for capsid proteins, polymerase enzyme, and envelope components, respectively. A sequence found upstream from the gag gene contains a signal for packaging of the genome into virions. Two long terminal repeat (LTR) sequences are present at the 5′ and 3′ ends of the viral genome. These contain strong promoter and enhancer sequences and are also required for integration in the host cell genome (Coffin, 1990).

In order to construct a retroviral vector, a nucleic acid encoding a gene of interest is inserted into the viral genome in the place of certain viral sequences to produce a virus that is replication-defective. In order to produce virions, a packaging cell line containing the gag, pol, and env genes but without the LTR and packaging components is constructed (Mann et al., 1983). When a recombinant plasmid containing a cDNA, together with the retroviral LTR and packaging sequences is introduced into this cell line (by calcium phosphate precipitation for example), the packaging sequence allows the RNA transcript of the recombinant plasmid to be packaged into viral particles, which are then secreted into the culture media (Nicolas and Rubenstein, 1988; Temin, 1986; Mann et al., 1983). The media containing the recombinant retroviruses is then collected, optionally concentrated, and used for gene transfer. Retroviral vectors are able to infect a broad variety of cell types. However, integration and stable expression require the division of host cells (Paskind et al., 1975).

A novel approach designed to allow specific targeting of retrovirus vectors was recently developed based on the chemical modification of a retrovirus by the chemical addition of lactose residues to the viral envelope. This modification could permit the specific infection of hepatocytes via sialoglycoprotein receptors.

A different approach to targeting of recombinant retroviruses was designed in which biotinylated antibodies against a retroviral envelope protein and against a specific cell receptor were used. The antibodies were coupled via the biotin components by using streptavidin (Roux et al., 1989). Using antibodies against major histocompatibility complex class I and class II antigens, they demonstrated the infection of a variety of human cells that bore those surface antigens with an ecotropic virus in vitro (Roux et al., 1989).

There are certain limitations to the use of retrovirus vectors in all aspects of the present invention. For example, retrovirus vectors usually integrate into random sites in the cell genome. This can lead to insertional mutagenesis through the interruption of host genes or through the insertion of viral regulatory sequences that can interfere with the function of flanking genes (Varmus et al., 1981). Another concern with the use of defective retrovirus vectors is the potential appearance of wild-type replication-competent virus in the packaging cells. This can result from recombination events in which the intact-sequence from the recombinant virus inserts upstream from the gag, pol, env sequence integrated in the host cell genome. However, new packaging cell lines are now available that should greatly decrease the likelihood of recombination (Markowitz et al., 1988; Hersdorffer et al., 1990).

Werner et al. (2004) describe B-cell specific transgene expression using a self-inactivating retroviral vector. Spleen Focus Forming Virus (SFFV) enhancer promoter or CD19 promoter were selected to direct expression of transgenes in hematopoietic cells following retroviral transfer. These vectors, termed SIN vectors for their self-inactivating properties, provided long-term in vivo expression (to at least one year).

C. Other Viral Vectors

Other viral vectors may be employed as expression constructs in the present invention. Vectors derived from viruses such as vaccinia virus (Ridgeway, 1988; Baichwal and Sugden, 1986; Coupar et al., 1988) adeno-associated virus (AAV) (Ridgeway, 1988; Baichwal and Sugden, 1986; Hermonat and Muzycska, 1984) and herpesviruses may be employed. They offer several attractive features for various mammalian cells (Friedmann, 1989; Ridgeway, 1988; Baichwal and Sugden, 1986; Coupar et al., 1988; Horwich et al., 1990).

Epstein-Barr virus, frequently referred to as EBV, is a member of the herpesvirus family and one of the most common human viruses. The virus occurs worldwide, and most people become infected with EBV sometime during their lives. In the United States, as many as 95% of adults between 35 and 40 years of age have been infected. When infection with EBV occurs during adolescence or young adulthood, it causes infectious mononucleosis 35% to 50% of the time. EBV vectors have been used to efficiently deliver DNA sequences to cells, in particular, to B lymphocytes. Robertson et al. (1986) provides a review of EBV as a gene therapy vector.

With the recognition of defective hepatitis B viruses, new insight was gained into the structure-function relationship of different viral sequences. In vitro studies showed that the virus could retain the ability for helper-dependent packaging and reverse transcription despite the deletion of up to 80% of its genome (Horwich et al., 1990). This suggested that large portions of the genome could be replaced with foreign genetic material. The hepatotropism and persistence (integration) were particularly attractive properties for liver-directed gene transfer. Chang et al., introduced the chloramphenicol acetyltransferase (CAT) gene into duck hepatitis B virus genome in the place of the polymerase, surface, and pre-surface coding sequences. It was co-transfected with wild-type virus into an avian hepatoma cell line. Culture media containing high titers of the recombinant virus were used to infect primary duckling hepatocytes. Stable CAT gene expression was detected for at least 24 days after transfection (Chang et al., 1991).

In order to effect expression of sense or antisense gene constructs, the expression construct must be delivered into a cell. This delivery may be accomplished in vitro, as in laboratory procedures for transforming cells lines, or in vivo or ex vivo, as in the treatment of certain disease states. One mechanism for delivery is via viral infection where the expression construct is encapsidated in an infectious viral particle.

5. Non-Viral Delivery

Several non-viral methods for the transfer of expression constructs into cultured mammalian cells also are contemplated by the present invention. These include calcium phosphate precipitation (Graham and Van Der Eb, 1973; Chen and Okayama, 1987; Rippe et al., 1990) DEAE-dextran (Gopal, 1985), electroporation (Tur-Kaspa et al., 1986; Potter et al., 1984), direct microinjection (Harland and Weintraub, 1985), DNA-loaded liposomes (Nicolau and Sene, 1982; Fraley et al., 1979) and lipofectamine-DNA complexes, cell sonication (Fechheimer et al., 1987), gene bombardment using high velocity microprojectiles (Yang et al., 1990), and receptor-mediated transfection (Wu and Wu, 1987; Wu and Wu, 1988). Some of these techniques may be successfully adapted for in vivo or ex vivo use.

In yet another embodiment of the invention, the expression construct may simply consist of naked recombinant DNA or plasmids. Transfer of the construct may be performed by any of the methods mentioned above which physically or chemically permeabilize the cell membrane. This is particularly applicable for transfer in vitro but it may be applied to in vivo use as well. Dubensky et al. (1984) successfully injected polyomavirus DNA in the form of calcium phosphate precipitates into liver and spleen of adult and newborn mice demonstrating active viral replication and acute infection. Benvenisty and Neshif (1986) also demonstrated that direct intraperitoneal injection of calcium phosphate-precipitated plasmids results in expression of the transfected genes. It is envisioned that DNA encoding a gene of interest may also be transferred in a similar manner in vivo and express the gene product.

In still another embodiment of the invention for transferring a naked DNA expression construct into cells may involve particle bombardment. This method depends on the ability to accelerate DNA-coated microprojectiles to a high velocity allowing them to pierce cell membranes and enter cells without killing them (Klein et al., 1987). Several devices for accelerating small particles have been developed. One such device relies on a high voltage discharge to generate an electrical current, which in turn provides the motive force (Yang et al., 1990). The microprojectiles used have consisted of biologically inert substances such as tungsten or gold beads.

Selected organs including the liver, skin, and muscle tissue of rats and mice have been bombarded in vivo (Yang et al., 1990; Zelenin et al., 1991). This may require surgical exposure of the tissue or cells, to eliminate any intervening tissue between the gun and the target organ, i.e., ex vivo treatment. Again, DNA encoding a particular gene may be delivered via this method and still be incorporated by the present invention.

In a further embodiment of the invention, the expression construct may be entrapped in a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). Also contemplated are lipofectamine-DNA complexes.

Liposome-mediated nucleic acid delivery and expression of foreign DNA in vitro has been very successful. Wong et al., (1980) demonstrated the feasibility of liposome-mediated delivery and expression of foreign DNA in cultured chick embryo, HeLa and hepatoma cells. Nicolau et al. (1987) accomplished successful liposome-mediated gene transfer in rats after intravenous injection.

In certain embodiments of the invention, the liposome may be complexed with a hemagglutinating virus (HVJ). This has been shown to facilitate fusion with the cell membrane and promote cell entry of liposome-encapsulated DNA (Kaneda et al., 1989). In other embodiments, the liposome may be complexed or employed in conjunction with nuclear non-histone chromosomal proteins (HMG-1) (Kato et al., 1991). In yet further embodiments, the liposome may be complexed or employed in conjunction with both HVJ and HMG-1. In that such expression constructs have been successfully employed in transfer and expression of nucleic acid in vitro and in vivo, then they are applicable for the present invention. Where a bacterial promoter is employed in the DNA construct, it also will be desirable to include within the liposome an appropriate bacterial polymerase.

Other expression constructs which can be employed to deliver a nucleic acid encoding a particular gene into cells are receptor-mediated delivery vehicles. These take advantage of the selective uptake of macromolecules by receptor-mediated endocytosis in ahnost all eukaryotic cells. Because of the cell type-specific distribution of various receptors, the delivery can be highly specific (Wu and Wu, 1993).

Receptor-mediated gene targeting vehicles generally consist of two components: a cell receptor-specific ligand and a DNA-binding agent. Several ligands have been used for receptor-mediated gene transfer. The most extensively characterized ligands are asialoorosomucoid (ASOR) (Wu and Wu, 1987) and transferrin (Wagner et al., 1990). Recently, a synthetic neoglycoprotein, which recognizes the same receptor as ASOR, has been used as a gene delivery vehicle (Ferkol et al., 1993; Perales et al., 1994) and epidermal growth factor (EGF) has also been used to deliver genes to squamous carcinoma cells (Myers, EPO 0273085).

In other embodiments, the delivery vehicle may comprise a ligand and a liposome. For example, Nicolau et al., (1987) employed lactosyl-ceramide, a galactose-terminal asialganglioside, incorporated into liposomes and observed an increase in the uptake of the insulin gene by hepatocytes. Thus, it is feasible that a nucleic acid encoding a particular gene also may be specifically delivered into a cell type by any number of receptor-ligand systems with or without liposomes. For example, epidermal growth factor (EGF) may be used as the receptor for mediated delivery of a nucleic acid into cells that exhibit upregulation of EGF receptor. Mannose can be used to target the mannose receptor on liver cells. Also, antibodies to CD5 (CLL), CD22 (lymphoma), CD25 (T-cell leukemia) and MAA (melanoma) can similarly be used as targeting moieties.

6. Recombination Events

A. Homologous Recombination

In one aspect of the invention, the Ig expression cassette are provided in a form that permits their integration at specifc sites in the host cell genome though the use of homologous recombination. Homologous recombination relies on the tendency of nucleic acids to base pair with complementary sequences. In this instance, the base pairing serves to facilitate the interaction of two separate nucleic acid molecules so that strand breakage and repair can take place. In other words, the “homologous” aspect of the method relies on sequence homology to bring two complementary sequences into close proximity, while the “recombination” aspect provides for one complementary sequence to replace the other by virtue of the breaking of certain bonds and the formation of others.

Put into practice, homologous recombination is used as follows. First, a target locus is selected within the host cell. Sequences homologous to the target are then included in a genetic construct, along with some additional sequences to be introduced. The homologous sequences are placed such that they flank the additional sequences. Flanking, in this context, simply means that target homologous sequences are located both upstream (5′) and downstream (3′) of the additional sequences. These sequences should correspond to sequences upstream and downstream regions of the target locus. The construct is then introduced into the cell, thus permitting recombination between the cellular sequences and the construct.

As a practical matter, the genetic construct will normally act as far more than a vehicle to introduce a sequence. For example, it is important to be able to select for recombinants and, therefore, it is common to include within the construct a selectable marker gene. This gene permits selection of cells that have integrated the construct into their genomic DNA by conferring resistance to various biostatic and biocidal drugs. An arrangement might be as follows:
. . . vector•5′-flanking sequence•additional sequences selectable marker gene•flanking sequence-3′•vector . . .
   Thus, using this kind of construct, it is possible, in a single recombinatorial event, to (i) “knock out” an endogenous gene, (ii) provide a selectable marker for identifying such an event and (iii) introduce a heterologous gene for expression.

Another refinement of the homologous recombination approach involves the use of a “negative” selectable marker. This marker, unlike the selectable marker, causes death of cells which express the marker. Thus, it is used to identify (and eliminate) undesirable recombination events. When seeking to select homologous recombinants using a selectable marker, it is difficult in the initial screening step to identify proper homologous recombinants from recombinants generated from random, non-sequence specific events. These recombinants also may contain the selectable marker gene and may express the heterologous protein of interest, but will, in all likelihood, not have the desired “knock out” phenotype. By attaching a negative selectable marker to the construct, but outside of the flanking regions, one can select against many random recombination events that will incorporate the negative selectable marker. Homologous recombination should not introduce the negative selectable marker, as it is outside of the flanking sequences. Thus, one possible arrangement of sequences would be:
. . . vector•negative selectable marker gene•5′-flanking target sequences•additional sequences•drug-selectable marker gene•flanking target sequences-3′•vector
   Of course, the negative selectable marker gene could come at the 3′-end of the construct and the additional sequences and drug-selectable marker genes could exchange positions. Site-specific recombination, relying on the homology between the vector and the target gene, will result in incorporation of the selected gene and the drug selectable marker gene only; the negative selectable marker sequences will not be introduced in the homologous recombination event because they lie outside the flanking sequences. These cells will be drug resistant and not acquire the negative selectable marker sequences and, thus, remain insensitive to selection. This double-selection procedure should yield recombinants that contain the lack the target gene and express the selected gene. Further screens for these phenotypes, either functional or immunologic, may be applied.

B. Random Integration

Though lacking the specificity of homologous recombination, there may be situations where random integration will be used as a method of introducing the Ig expression cassettes of the present invention. Unlike homologous recombination, the recombinatorial event here is completely random, i.e., not reliant upon base-pairing of complementary nucleic acid sequences. Random integration is like homologous recombination, however, in that a gene construct integrates into the target cell genomic DNA via strand breakage and reformation.

Because of the lack of sequence specificity, the chances of any given recombinant integrating into the target gene are greatly reduced. Also possible is integration into a second loci, resulting in the loss of expression of an important host cell gene, or the masking of expression of the additional sequences to be inserted. As a result, it may be necessary to “brute force” the selection process, in other words, to screen hundreds of thousands of drug-resistant recombinants before a desired cell is found. Screening can be facilitated, for example, by examining recombinants for expression of the additional sequences using immunologic or even functional tests.

7. Ex Vivo Delivery

In certain embodiments, gene transfer may more easily be performed under ex vivo conditions. Ex vivo gene therapy refers to the isolation of cells from an animal, the delivery of a nucleic acid into the cells in vitro, and then the return of the modified cells back into an animal. This involves the removal of cells or tissues from an animal, and/or the primary culture of removed cells or tissues.

In the present invention, the cell type of interest is a human lymphocytic progenitor cells that can progress to an immunoglobuin-producing cell (i.e., B cell). These cells may be obtained from human bone marrow or cord blood samples preserved at birth.

Bone marrow sampling can be performed by a hematologist, internist or by a specially-trained technologist. A laboratory technologist may also help prepare the sample. Blood samples may be collected before the test. Rarely, blood clotting factors may be given to prevent prolonged bleeding.

Adults usually have a sample of bone marrow fluid taken from the back of the hipbone (posterior ilium). Rarely, a fluid sample is removed from the breastbone (sternum) or from the front of the hipbone (anterior iliac crest). Infants and young children may have the sample taken from the front of the lower leg bone (tibia), just below the knee.

The patient will lie either on their side or abdomen while the health professional obtains a bone marrow aspiration and biopsy. The skin over the biopsy site will be cleaned with an antiseptic solution and a local anesthetic will be injected to numb the area. The biopsy needle is inserted through the skin and into bone to reach the bone marrow. A sample of the marrow is drawn through a needle into a syringe. A solid form of bone marrow may be collected with the same needle or another needle (a biopsy of a solid form of bone marrow is generally taken from the hipbone). The needle is then withdrawn. More than one sample may be needed, possibly from more than one site, such as both hipbones for a bone marrow harvest. After the samples have been collected, pressure is applied to help stop any bleeding and a bandage is applied to the site.

Each sampling takes about 10 to 20 minutes. After the test, the patient should remain prone for 10 to 15 minutes. If the bleeding has stopped at the end of that time, the patient may resume normal activities.

Subsequently, it may be necessary to culture the cells obtained from the patient, either for expansion or for conditioning prior to transformation. In one study, it was shown that incubation of post-fluorouracil bone marrow cells in WEHI-3 CM for 7 days resulted in a 60-fold increase of primitive progenitor cells (13-day spleen colony-forming units) and a 53-fold increase in committed progenitor cells (granulocyte-macrophage colony-forming cells; GM-CFC) (Bradley et al., 1985). In subsequent studies from the same group, it was shown that preincubation with HGFs (also using crude CM) could expand primitive murine progenitor cells (HPP-CFC) and cells with in vivo marrow repopulating ability (McNiece et al., 1986; McNiece et al., 1987). Using a similar culture system of human bone marrow cells in Teflon bottles, it has been shown that the combination of recombinant human granulocyte-macrophage colony-stimulating factor (rhGM-CSF) plus recombinant human interleukin-3 (rhIL-3) could generate a 7-fold increase in committed progenitor cells (GM-CFC) (McNiece et al., 1988). In 1991, Bernstein et al. (1991) showed that incubation of single CD34+Lin-cells in the combination of IL-3, granulocyte colony-stimulating factor (G-CSF), and stem cell factor (SCF) gave rise to a 10-fold increase of colonies in vitro.

The use of ex vivo expansion to generate mature neutrophil precursors was proposed by Haylock et al. (1992). These authors demonstrated that the combination of IL-1, IL-3, IL-6, GM-CSF, and SCF could generate a 1324-fold increase in nucleated cells, and a 66-fold increase in GM-CFC. The cells produced under these conditions were predominantly neutrophil precursors. The culture conditions used were static and used CD34+ cells as the starting population. Several investigators have demonstrated the requirement for CD34 selection of the starting cells for optimal expansion. Subsequent studies were performed on a clinical scale using optimal culture conditions in Teflon bags with fully defined media appropriate for clinical applications. This work used the growth factor cocktail comprising SCF, G-CSF, and megakaryocyte growth and development factor (MGDF). Other cocktails of growth factors are effective in expanding CD34+ cells; however, the availability of clinical-grade growth factors has been limited due to commercial considerations.

The in vivo potential of ex vivo expanded cells was first reported in murine studies by Muench et al. (1993). This study demonstrated that bone marrow cells expanded in SCF plus IL-1 engrafted lethally irradiated mice and were capable of sustaining long-term hematopoiesis in these animals. In addition, the bone marrow from these engrafted mice could repopulate secondary recipients. The authors concluded that the expansion of mouse bone marrow cells did not adversely affect the proliferative capacity and lineage potential of the stem cell compartment.

IV. Treating Agammaglobulinemias

A. Primary Agammaglobulinemias

Three main types of primary agamnimaglobulinemias or “hypogammaglobulinemias” exist: X-linked aganimaglobulinemia, X-linked agammaglobulinemia with growth hormone deficiency, and autosomal recessive agammaglobulinemia. Each of these diseases is characterized by the reduction or absence of Ig production, due to defects in B cell development, and sometimes T cell development as well.

X-linked agammaglobulinemia, also called “Bruton's Disease,” is responsible for about 50% of all cases. Named in honor of Bruton, who first reported the disease in 1952, the causative agent has been identified as a defect in Bruton's tyrosine kinase, or Btk. Recently, defective antibody production and low B cell numbers have been described in female infants and males in whom no Btk abnormalities were detected, thereby implicating the involvement of other genes.

X-linked hypogammaglobulinemia with growth hormone deficiency was first described by Fleisher et al. (1980). The defect has been mapped to the same region that encompasses Btk gene and may involve a gene controlling growth hormone production (Raynaud, 1998), implying a small contiguous gene deletion that includes both the gene for XLA and another closely linked gene involved in growth hormone production.

In addition to the genetic defects described above, other pathophysiology mechanisms may result in hypogammaglobulinemia or agammaglobulinemia, such as viral infections, malignancy, or drug effects.

Defects may occur at a variety of points in the development and maturation of B-cells, resulting in the lack of Ig production. In the fetal bone marrow, the first committed cell in B-cell development is the early pro-B cell identified by its ability to proliferate in the presence of IL-7 (Kee and Murre, 1998). These cells develop into late pro-B cells in which rearrangement of the heavy chain occurs. This rearrangement process requires the recombination activating genes, which are controlled by various factors (IL-7 in the mouse). Once the heavy chain is produced, it is transported to the cell surface.

Progression from late pro-B-cell to the pre-B-cell stage involves the rearrangement and joining of the various segments of the heavy chain. The completion of rearrangement of the light and heavy chains and the presence of surface IgM results in the immature B cell, which then leaves the bone marrow. Increasing levels of immunoglobulin D (IgD) finally results in the mature B cells expressing both IgM and IgD. T cells, along with further stimuli and various chemokines, stimulate B cells to undergo further proliferation and Ig class switching, leading to the expression of the various isotypes IgG, IgA, or immunoglobulin E (IgE).

B. Combined Therapy

In another embodiment, it is envisioned that transfer of strong Ig promoter constructs may be performed in combination with other therapeutic modalities. Thus, in addition to the therapies described above, one may also provide to the patient more “standard” therapies for agammaglobulinemia, which is primarily passive immune therapy., supplemented with aggressive antibiotic treatment for bacterial infections.

Intravenous delivery of Ig (IVIG) results in improved clinical status with a decrease in infections like pneumonia and meningitis. Patients who receive high-dose IVIG (400-500 mg/kg q3-4 wk) and who maintained IgG levels higher than 500 mg/dL had fewer hospitalizations and infections. Generally, the goal is to maintain a trough serum IgG level of at least 500 mg/dL. However, in practice the patient with an endpoint being fewer infections. This may involve higher doses and/or more frequent infusions. Because of the blood brain barrier, patients with viral meningitis require 1000 mg/kg.

In patients with chronic respiratory infections, long-term broad-spectrum antibiotics may be needed, in addition to chest physiotherapy and sinus surgery. Specific antibiotic choices must cover the usual polysaccharide-encapsulated organisms, and higher doses and longer courses are common.

Some patients develop chronic sinusitis despite regular IVIG replacement therapy. These patients are challenging to treat because antibiotics, N-acetylcysteine, and topical intranasal corticosteroid therapies fail to clear pathogens and do not decrease sinus inflammation. Because of the possible development of chronic sinusitis, surgical intervention may be required to promote sinus drainage.

Antibody/drug combinations with the promoter replacement therapy of the present invention may be achieved by contacting cells with a single composition or pharmacological formulation that includes both agents, or by contacting the cell with two distinct compositions or formulations, at the same time. More likely, the promoter replacement therapy will precede and/or follow administration of the other agent(s) by intervals ranging from minutes to weeks. In embodiments where the agents are applied separately to the cell, one would generally ensure that a significant period of time did not expire between the time of each delivery, such that the agents would still be able to exert an advantageously combined effect on the cell. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

It also is likely that more than one administration of either the promoter replacement therapy or the other agent will be desired. In this regard, various combinations may be employed. By way of illustration, where the provision of promoter replacement according to the present invention is “A,” and the other agent is “B,” the following permutations based on 3 and 4 total administrations are exemplary:

A/B/A B/A/B B/B/A A/A/B B/A/A A/B/B B/B/B/A
B/B/A/B A/A/B/B A/B/A/B A/B/B/A B/B/A/A B/A/B/A
B/A/A/B B/B/B/A A/A/A/B B/A/A/A A/B/A/A A/A/B/A
A/B/B/B B/A/B/B B/B/A/B

Other combinations are likewise contemplated.

C. Therapeutic Agents

Pharmacological therapeutic agents such as expression constructs, cells, and immunoglobulins, as well as methods of administration, dosages, etc., are well known to those of skill in the art (see for example, the “Physicians Desk Reference,” Goodman and Gilman's “The Pharmacological Basis of Therapeutics,” “Remington's Pharmaceutical Sciences,” and “The Merck Index, Thirteenth Edition,” incorporated herein by reference in relevant parts), and may be combined with the invention in light of the disclosures herein. Some variation in dosage will necessarily occur depending on the condition of the subject being treated. The person responsible for administration will, in any event, determine the appropriate dose for the individual subject, and such individual determinations are within the skill of those of ordinary skill in the art.

It will be understood that in the discussion of formulations and methods of treatment, references to any compounds are meant to also include the pharmaceutically acceptable salts, as well as pharmaceutical compositions. Where clinical applications are contemplated, pharmaceutical compositions will be prepared in a form appropriate for the intended application. Generally, this will entail preparing compositions that are essentially free of pyrogens, as well as other impurities that could be harmful to humans or animals.

One will generally desire to employ appropriate salts and buffers to render delivery vectors stable and allow for uptake by target cells. Buffers also will be employed when recombinant cells are introduced into a patient. Aqueous compositions of the present invention comprise an effective amount of the vector or cells, dissolved or dispersed in a pharmaceutically acceptable carrier or aqueous medium. The phrase “pharmaceutically or pharmacologically acceptable” refer to molecular entities and compositions that do not produce adverse, allergic, or other untoward reactions when administered to an animal or a human. As used herein, “pharmaceutically acceptable carrier” includes solvents, buffers, solutions, dispersion media, coatings, antibacterial and antifungal agents, isotonic and absorption delaying agents and the like acceptable for use in formulating pharmaceuticals, such as pharmaceuticals suitable for administration to humans. The use of such media and agents for pharmaceutically active substances is well known in the art. Except insofar as any conventional media or agent is incompatible with the active ingredients of the present invention, its use in therapeutic compositions is contemplated. Supplementary active ingredients also can be incorporated into the compositions, provided they do not inactivate the vectors or cells of the compositions.

In specific embodiments of the invention the pharmaceutical formulation will be formulated for delivery via rapid release, other embodiments contemplated include but are not limited to timed release, delayed release, and sustained release. Formulations can be an oral suspension in either the solid or liquid form. In further embodiments, it is contemplated that the formulation can be prepared for delivery via parenteral delivery, or be formulated for subcutaneous, intravenous, intramuscular, intraperitoneal, transdermal, or nasopharyngeal delivery.

Aqueous suspensions contain an active material in admixture with excipients suitable for the manufacture of aqueous suspensions. Such excipients are suspending agents, for example sodium carboxymethylcellulose, methylcellulose, hydroxy-propylmethycellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethylene-oxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate, one or more coloring agents, one or more flavoring agents, and one or more sweetening agents, such as sucrose, saccharin or aspartame.

Oily suspensions may be formulated by suspending the active ingredient in a vegetable oil, for example arachis oil, olive oil, sesame oil or coconut oil, or in mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Pharmaceutical compositions may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring phosphatides, for example soy bean, lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening and flavouring agents.

The amount of active ingredient in any formulation may vary to produce a dosage form that will depend on the particular treatment and mode of administration. It is further understood that specific dosing for a patient will depend upon a variety of factors including age, body weight, general health, sex, diet, time of administration, route of administration, rate of excretion, drug combination and the severity of the particular disease undergoing therapy.

V. Examples

The following examples are included to further illustrate various aspects of the invention. It should be appreciated by those of skill in the art that the techniques disclosed in the examples that follow represent techniques and/or compositions discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLE 1

As discussed, most human immunoglobulin promoters do not have a consensus TATA box, whereas most mouse immunoglobulin genes have a good TATA box. Others have shown that the transcription factor TFII-I enhances transcription of “TATA-less” promoters such as those in the human Ig locus. The mouse Ig promoter that the inventors used for the study of Bright activity is a TATA-less promoter and regulates an immunoglobulin gene that is not effectively expressed in xid mice. Similarly, the homologous human Ig response is lacking in XLA patients.

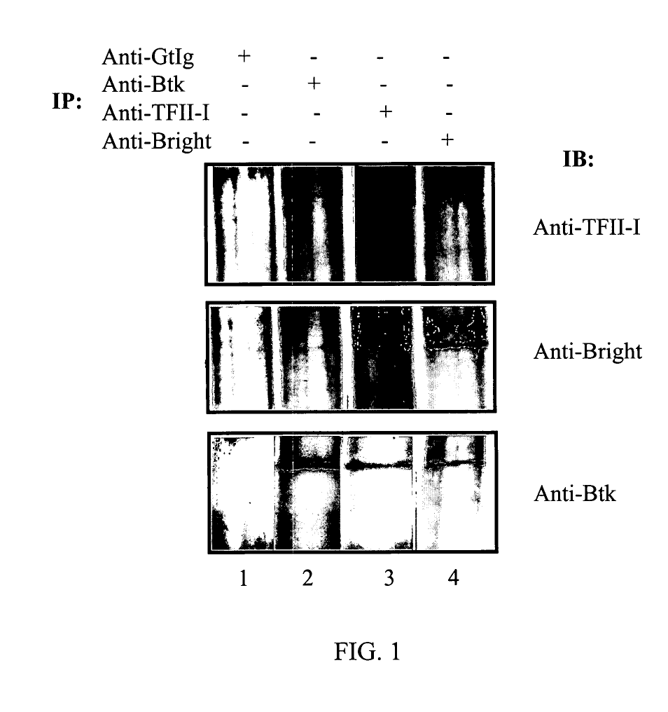

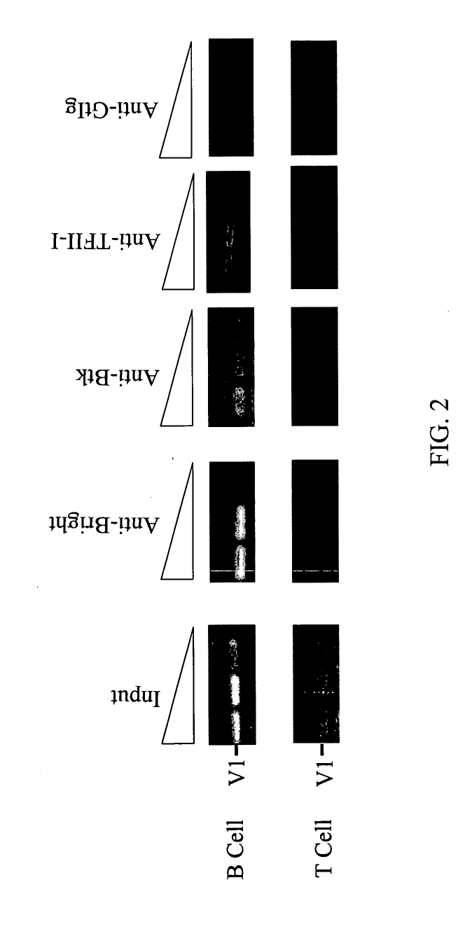

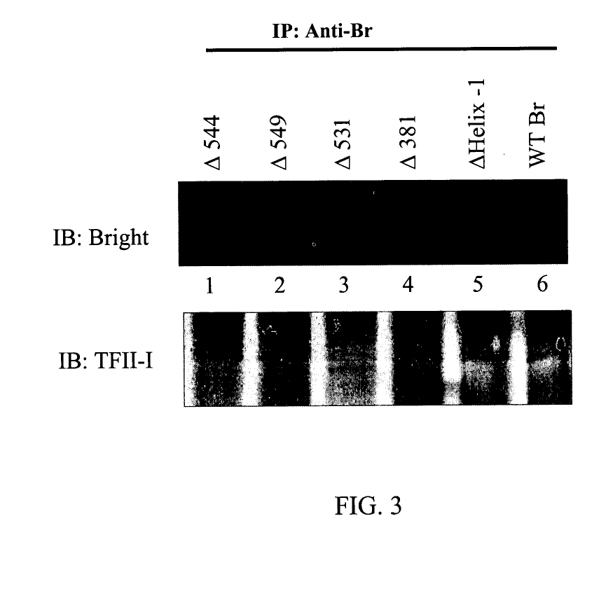

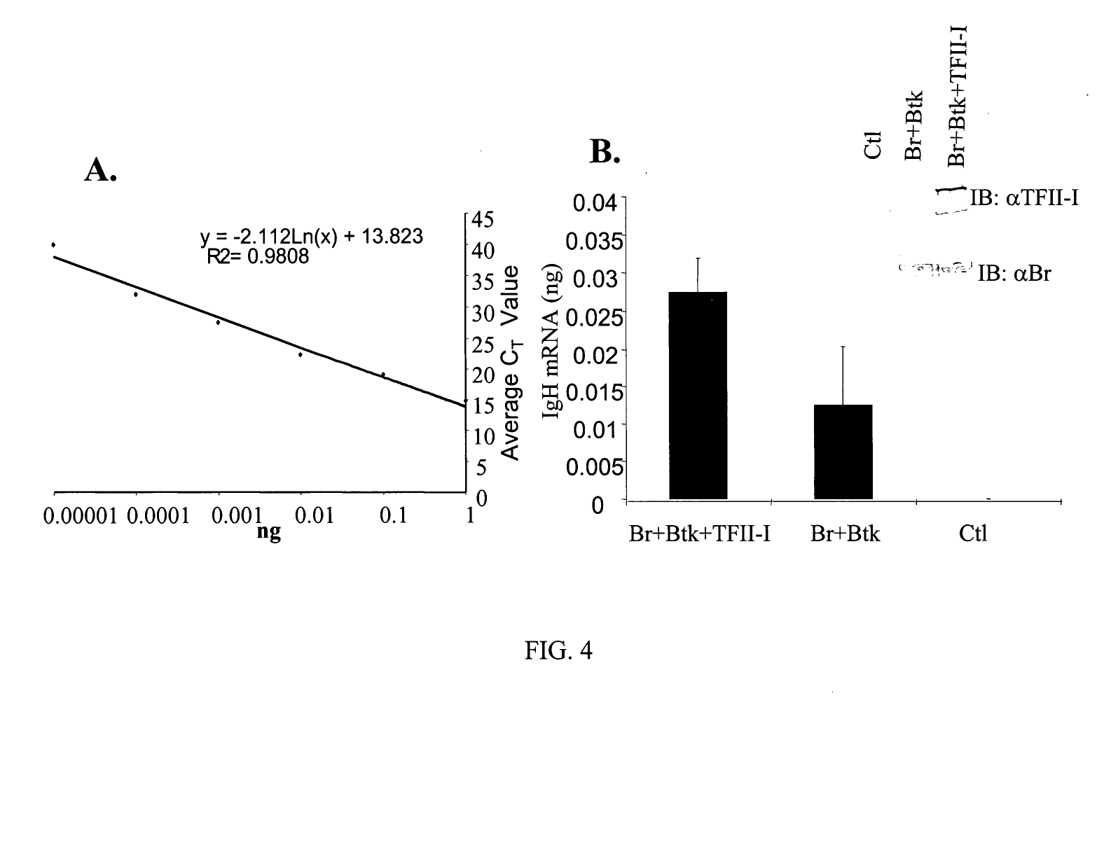

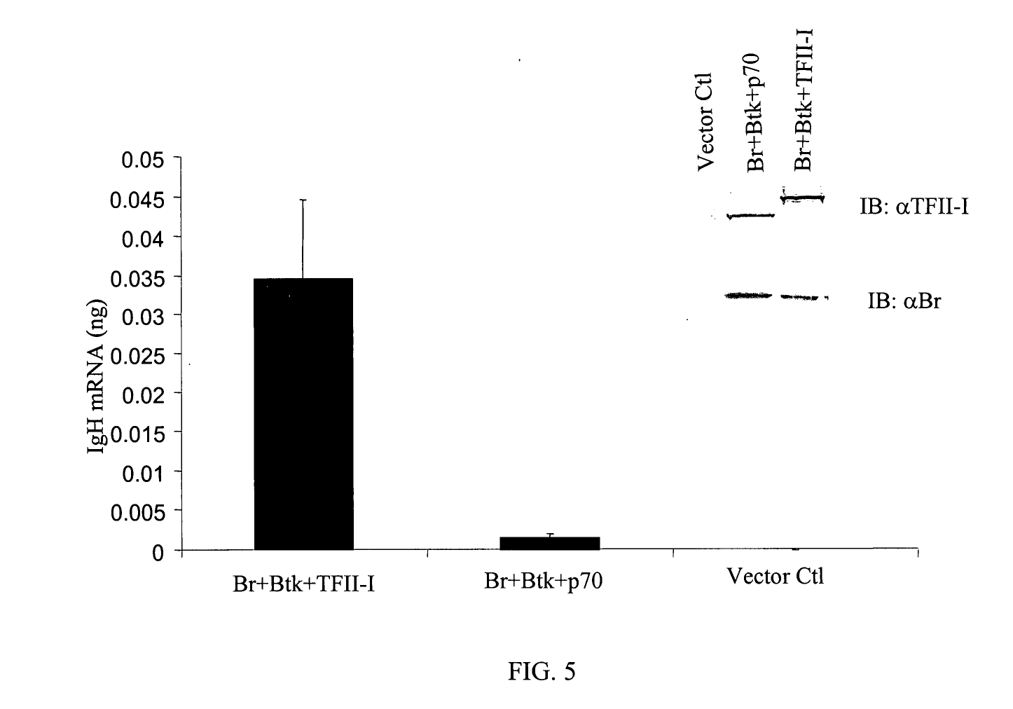

The data show that Bright-dependent activation of the mouse Ig gene critically requires TFII-I. Indeed, the inventors have evidence that demonstrates that Bright, Bruton's tyrosine kinase and TFII-I form a DNA-binding complex on that mouse promoter. (FIGS. 1-5). Therefore, the data strongly support the notion that some Ig genes differ in their requirements for promoter binding elements. The inventors have already shown that all Ig promoters do not have associated Bright binding sites.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods, and in the steps or in the sequence of steps of the methods described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

VI. References

The following references, to the extent that they provide exemplary procedural or other details supplementary to those set forth herein, are specifically incorporated herein by reference.
U.S. Pat. No. 4,166,452
U.S. Pat. No. 4,256,108
U.S. Pat. No. 4,265,874
Baichwal and Sugden, In: Gene Transfer, Kucherlapati (Ed.), NY, Plenum Press, 117-148, 1986.
Benvenisty and Neshif, Proc. Natl. Acad. Sci. USA, 83(24):9551-9555, 1986.
Bernstein et al., Exp. Hematol., 19(7):680-682, 1991.
Bradley et al., Prog. Clin. Biol. Res., 184:39-56, 1985.
Buchanan et al., J. Immunol., 155:4270-4277, 1995.
Buchanan et al., Immunology, 159:1247-1254, 1997.
Burns and Peterson, Mol. Cell. Biol., 17:4811-4819, 1997.
Chang et al., Hepatology, 14:134A, 1991.
Chen and Okayama, Mol. Cell Biol., 7(8):2745-2752, 1987.
Coffin, In: Virology, Fields et al. (Eds.), Raven Press, NY, 1437-1500, 1990.
Conley et al., Immunol. Rev., 138:5-21, 1994.
Couch et al., Am. Rev. Resp. Dis., 88:394-403, 1963.
Coupar et al., Gene, 68:1-10, 1988.
Dallas et al., Mol. Cell. Biol., 20:3137-3146, 2000.
Dubensky et al., Proc. Natl. Acad. Sci. USA, 81:7529-7533, 1984.
European Appln. EPO 0273085
Fattaey et al., Oncogene, 8:3149-3156, 1993.
Fechheimer et al., Proc Natl. Acad. Sci. USA, 84:8463-8467, 1987.
Ferkol et al., FASEB J, 7:1081-1091, 1993.
Fleisher et al., N. Engl. J. Med., 302(26):1429-1434, 1980.
Fraley et al., Proc. Natl. Acad. Sci. USA, 76:3348-3352, 1979.
Friedmann, Science, 244:1275-1281, 1989.
Ghosh and Bachhawat, In: Liver Diseases, Targeted Diagnosis and Therapy Using Specific Receptors and Ligands, Wu et al. (Eds.), Marcel Dekker, NY, 87-104, 1991.
Ghosh-Choudhury et al., EMBO J, 6:1733-1739, 1987.
Gomez-Foix et al., J. Biol. Chem., 267:25129-25134, 1992.
Goodman and Gilman's The Pharmacological Basis Of Therapeutics, Hardman et al. (Eds.), 10^{th} Ed., 32:853-860; 35:891-893, 2001.
Gopal, Mol. Cell Biol., 5:1188-1190, 1985.
Graham and Prevec, In: Methods in Molecular Biology: Gene Transfer and Expression Protocol, Murray (Ed.), Humana Press, Clifton, N.J., 7:109-128, 1991.
Graham and Van Der Eb, Virology, 52:456-467, 1973.
Graham et al., J. General Virology, 36:59-74, 1977.
Gregory et al., Mol. Cell. Biol. 16:792-799, 1996.
Grunhaus and Horwitz, Seminar in Virology, 3:237-252, 1992.
Harland and Weintraub, J. Cell Biol., 101(3):1094-1099, 1985.
Haylock et al., Blood, 80(6):1405-1412, 1992.
Hermonat and Muzycska, Proc. Natl. Acad. Sci. USA, 81:6466-6470, 1984.
Herrscher et al., Genes Dev. 9:3067-3082, 1995.
Hersdorffer et al., DNA Cell Biol., 9:713-723, 1990.
Herz and Gerard, Proc. Natl. Acad. Sci. USA, 90:2812-2816, 1993.
Horwich et al. J. Virol., 64:642-650, 1990.
Inaba et al., J. Exp. Med., 188:2163-2173, 1998.
Jones and Shenk, Cell, 13:181-188, 1978.
Kaneda et al., Science, 243:375-378, 1989.
Karlsson et al., EMBO J., 5:2377-2385, 1986.
Kato et al., J. Biol. Chem., 266:3361-3364, 1991.
Kee and Murre, J. Exp. Med., 188(4):699-713, 1998.
Klein et al., Nature, 327:70-73, 1987.
Kortschak et al., Genomics, 51:288-292, 1998.
Le Gal La Salle et al., Science, 259:988-990, 1993.
Levrero et al., Gene, 101: 195-202, 1991.
Mann et al., Cell, 33:153-159, 1983.
Markowitz et al., J. Virol., 62:1120-1124, 1988.
Matsuda et al., J. Exp. Med., 188(11):2151-2156, 1998.
McNiece et al., Exp. Hematol., 14(9):856-860, 1986.
McNiece et al., Exp. Hematol., 15(9):972-977, 1987.
McNiece et al., Exp. Hematol., 16(9):807-810, 1988.
Muench et al., Blood, 81(12):3463-3473, 1993.
Nicolas and Rubenstein, In: Vectors: A survey of molecular cloning vectors and their uses, Rodriguez and Denhardt (Eds.), Stoneham: Butterworth, 494-513, 1988.
Nicolau and Sene, Biochim. Biophys. Acta, 721:185-190, 1982.
Nicolau et al., Methods Enzymol., 149:157-176, 1987.
Numata et al., Cancer Res., 59:3741-3747, 1999.
Paskind et al., Virology, 67:242-248, 1975.
Perales et al., Proc. Natl. Acad. Sci. USA, 91:4086-4090, 1994.
Peterson and Herskowitz, Cell, 68:573-583, 1992.
Physicians Desk Reference
Potter et al., Proc. Natl. Acad. Sci. USA, 81:7161-7165, 1984.
Racher et al., Biotechnology Techniques, 9:169-174, 1995.
Ragot et al., Nature, 361:647-650, 1993.
Raynaud et al., Am. J. Med. Genet., 76(3):255-261, 1998.
Remington's Pharmaceutical Sciences, 15^{th} ed., pages 1035-1038 and 1570-1580, Mack Publishing Company, Easton, Pa., 1980.
Renan, Radiother. Oncol., 19:197-218, 1990.
Rich et al., Hum. Gene Ther., 4:461-476, 1993.
Ridgeway, In: Vectors: A survey of molecular cloning vectors and their uses, Rodriguez and Denhardt (Eds.), Stoneham:Butterworth, 467-492, 1988.
Rippe et al., Mol. Cell Biol., 10:689-695, 1990.
Robertson et al., Nature 322:445-448, 1986.
Rosenfeld et al., Science, 252:431-434, 1991.
Rosenfeld et al., Cell, 68:143-155, 1992.
Roux et al., Proc. Natl. Acad. Sci. USA, 86:9079-9083, 1989.
Satterthwaite and Witte, Ann. Rev. Immunol., 14:131-154, 1996.
Stratford-Perricaudet and Perricaudet, In: Human Gene Transfer, Eds, Cohen-Haguenauer and Boiron, John Libbey Eurotext, France, 51-61, 1991.
Stratford-Perricaudet et al., Hum. Gene. Ther., 1:241-256, 1990.
Temin, In: Gene Transfer, Kucherlapati (Ed.), NY, Plenum Press, 149-188, 1986.
The Merck Index, O'Neil et al., ed., 13^{th} ed., 2001.
Top et al., J. Infect. Dis., 124:155-160, 1971.
Treisman et al., Genes Dev. 11:1949-1962, 1997.
Tuaillon et al., Proc. Natl. Acad. Sci. USA, 90(8):3720-3724. 1993.
Tur-Kaspa et al., Mol. Cell Biol., 6:716-718, 1986.
Varmus et al., Cell, 25:23-36, 1981.
Wagner et al., Proc. Natl. Acad. Sci. USA 87(9):3410-3414, 1990.
Wang et al., Mol. Cell. Biol., 19:284-295, 1999.
Webb et al., Biochemistry, 28:4785-4790, 1989.
Webb et al., In: Differential regulation of immunoglobulin gene transcription via nuclear matrix-associated regions, Cold Spring Harbor Symp. Quant. Biol. LXIV, 109, 1999.
Webb et al., J. Immunol. 160:4747-4754, 1998.
Webb et al., J. Immunol., 165:6956-6965, 2000.
Webb et al., J. Immunol., 143:3934-3939, 1989.
Webb et al., Mol. Cell. Biol., 11:5197-5205, 1991.
Webb et al., Mol. Cell. Biol., 11:5206-5211, 1991.
Werner et al., Gene Ther., 11(12):992-100, 2004.
Wong et al., Gene, 10:87-94, 1980.
Wu and Wu, Adv. Drug Delivery Rev., 12:159-167, 1993.
Wu and Wu, Biochemistry, 27:887-892, 1988.
Wu and Wu, J. Biol. Chem., 262:4429-4432, 1987.
Yang et al., Proc. Natl. Acad. Sci. USA, 87:9568-9572, 1990.
Zelenin et al., FEBS Lett., 280:94-96, 1991.

What is claimed is:

1. A human Ig transcription cassette comprising, in a 5′ to 3′ arrangement:
(a) a promoter comprising a TATA element;
(b) at least two human Ig variable heavy (V_{H}) segments;
(c) at least two human Ig diversity (D) segments;
(d) at least two human Ig heavy joining (J_{H}) segments; and
(e) a human Ig constant heavy (CH) segment.

2. The transcription cassette of claim 1, comprising ten D segments.

3. The transcription cassette of claim 1, comprising six J_{H} segments.

4. The transcription cassette of claim 1, wherein said C_{H} segment is Cμ or Cγ.

5. The transcription cassette of claim 1, wherein said promoter is a murine Ig promoter.

6. The transcription cassette of claim 5, wherein said murine Ig promoter is a J558 family promoter.

7. The transcription cassette of claim 1, wherein said transcription cassette is comprised within a vector.

8. The transcription cassette of claim 7, wherein said vector is a non-viral vector.

9. The transcription cassette of claim 8, wherein said non-viral vector is a plasmid, a phagemid or a cosmid.

10. The transcription cassette of claim 7, wherein said vector is a viral vector.

11. The transcription cassette of claim 10, wherein said viral vector is an adenoviral vector, a retroviral vector, an adeno-associated viral vector, a herpesviral vector, or a vaccinia viral vector.

12. The transcription cassette of claim 1, further comprising a transcription termination signal.

13. The transcription cassette of claim 1, further comprising a selectable or screenable marker segment operably linked to said promoter.

14. A method of converting a human lymphocytic progenitor cell into a B cell comprising transforming said B cell with a first transcription cassette comprising, in a 5′ to 3′ arrangement:
(a) a promoter comprising a TATA element;
(b) at least two human Ig variable heavy (V_{H}) segments;
(c) at least two human Ig diversity (D) segments;
(d) at least two human Ig heavy joining (J_{H}) segments; and
(e) a human Ig constant heavy (C_{H}) segment.

15. The method of claim 14, wherein transferring comprises homologous recombination of said transcription cassette into the genome of said lymphocytic progenitor cell.

16. The method of claim 14, wherein said lymphocytic progenitor cell is obtained from a human subject prior to transforming, and is reintroduced into said human subject after transforming.

17. The method of claim 16, wherein said human subject suffers from primary agammaglobulinemia.

18. The method of claim 17, wherein said primary agammaglobulinemia is X-linked agammaglobulinemia, X-linked agammaglobulinemia with growth hormone deficiency, and autosomal recessive agammaglobulinemia.

19. The method of claim 16, wherein said lymphocytic progenitor cell is obtained from cord blood, or bone marrow.

20. The method of claim 14, further comprising transforming said lymphocytic progenitor cell with a second transcription cassette comprising, in a 5′ to 3′ arrangement:
(a) a promoter comprising a TATA element;
(b) at least two human Ig variable heavy (V_{H}) segments;
(c) at least two human Ig diversity (D) segments;
(d) at least two human Ig heavy joining (J_{H}) segments; and
(e) a human Ig constant heavy (CH) segment,
wherein said V_{H} segments are distinct from those in said first transcription cassette.

21. The method of claim 14, wherein said lymphocytic progenitor cell is obtained from cord blood or bone marrow of one subject and is introduced, after transformation, into a genetically-related subject.
